(19) United States
(12) Patent Application Publication
(10) Pub. No.: US 2021/0275443 A1
(43) Pub. Date: Sep. 9, 2021

(54) SUSTAINED-RELEASE INJECTION PREPARATION CONTAINING DONEPEZIL DERIVATIVE

(71) Applicant: NANJING NORATECH PHARMACEUTICAL CO., LTD., Nanjing, Jiangsu (CN)

(72) Inventors: Cuixia ZHANG, Nanjing, Jiangsu (CN); Fei LIU, Nanjing, Jiangsu (CN); Wei LIU, Nanjing, Jiangsu (CN); Lulu WANG, Nanjing, Jiangsu (CN); Deyan YIN, Nanjing, Jiangsu (CN)

(21) Appl. No.: 17/261,379

(22) PCT Filed: May 30, 2019
(86) PCT No.: PCT/CN2019/089370

§ 371 Date: Jan. 19, 2021

(30) Foreign Application Priority Data
May 30, 2018 (CN) 201810536059.0

(51) Int. Cl. A61K 9/00; A61K 9/08; A61K 31/445; A61K 47/02; A61K 47/26

(52) U.S. Cl. CPC A61K 9/0019; A61K 9/08; A61K 47/26; A61K 47/02; A61K 31/445

(57) ABSTRACT

Disclosed are a sustained-release injection preparation containing a donepezil derivative in the form of a suspension, and the use of the preparation in the preparation of a medicament for treating diseases caused by abnormal acetylcholinesterase activity.

### TECHNICAL FIELD

The present invention relates to the field of pharmaceutical preparation. In particular the present invention relates to a sustained-release injection preparation in the form of a suspension, and uses thereof in the preparation of a medicament for treating diseases caused by abnormal acetylcholinesterase activity.

### BACKGROUND

Donepezil is a central acetylcholinesterase inhibitor developed by Eisai Pharmaceutical Co., Ltd (Japan). It was first sold in the United States in 1997 and in China in 1999, and is currently sold in many countries around the world. Donepezil has advantages of small dosage, low toxicity and low cost. It is the first choice for treating mild to moderate Alzheimer's disease (AD). In addition, it can also be used to treat memory dysfunction, reduce memory loss, dry mouth, and constipation in patients with non-senile affective disorder. It can also be used to treat diseases, such as purpuric rash, vascular dementia, and sleep behavior disorder in rapid eye movement sleep phases.

Donepezil has good water solubility. The conventional preparation of Donepezil is a film-coated tablet with good oral absorption and high bioavailability. However, it needs to be taken every day and has a long taking period. After administration, the blood concentration of Donepezil rises rapidly, which may cause adverse reactions, such as vomiting, diarrhea or insomnia and cause great pain and inconvenience to patients. Moreover, patients with Alzheimer's have poor memory, therefore they often forget to take medicines, or take the wrong medicines. And there are some AD patients who often refuse to admit that they are sick so as to not take medicines, thereby resulting in poor compliance, a high clinical loss rate and seriously affecting the therapeutic effects.

Therefore, it is necessary to develop a sustained-release injection preparation, which will possess certain stability and can be stored for a long time. Upon administration, the content of Donepezil in plasma can be maintained within the effective blood concentration range for a long time, thereby significantly prolonging the efficacy, reducing the frequency of administration, improving the patient compliance and avoiding side effects caused by rapid increase in blood concentration.

### SUMMARY OF THE INVENTION

An objective of the present invention is to provide a sustained-release injection preparation in the form of a suspension of donepezil derivatives, and its use in the preparation of a medicament for treating diseases caused by abnormal acetylcholinesterase activity. The diseases include Alzheimer's disease.

The objectives of the present invention is achieved through following technical solutions:

In the present invention, a sustained-release injection preparation in the form of a suspension is provided, wherein the injection preparation comprises a compound of formula (I), a wetting agent and a suspending agent,

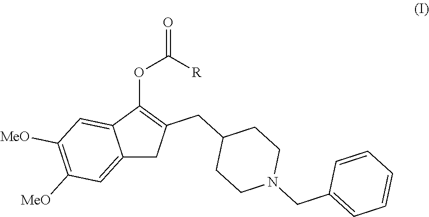

wherein:

R is a C₅₋₁₇ alkyl.

In one embodiment, the wetting agent in the sustained-release injectable formulation of the present invention is one or more selected from the group consisting of polysorbates, polyoxyethylene castor oils, poloxamers, tylosamers, povidone, lecithin, polyoxyethylene hydrogenated castor oil, bile salt and polyoxyethylene polyoxypropylene ether block copolymer; and preferably one or more selected from the group consisting of polysorbate 20, polysorbate 40 and polysorbate 80; and more preferably, polysorbate 80.

In one embodiment, the suspending agent in the sustained-release injection preparation of the present invention is selected from natural polymer suspending agents, synthetic or semi-synthetic polymer suspending agents; preferably one or more selected from the group consisting of gums, celluloses, carbopol, povidone, dextran and polyethylene glycol; more preferably one or more selected from the group consisting of sodium carboxymethyl cellulose (CMC-Na), methyl cellulose, hydroxypropyl cellulose, gum arabic, xanthan gum, povidone and polyethylene glycol; and most preferably, one or more selected from the group consisting of polyethylene glycol 200, polyethylene glycol 400, polyethylene glycol 600, polyethylene glycol 800, polyethylene glycol 1000, polyethylene glycol 1500, polyethylene glycol 4000, polyethylene glycol 6000 and polyethylene glycol 8000.

In one embodiment, the mixture ratio (weight) of the wetting agent to the suspending agent in the sustained-release injection preparation of the present invention is 1:0.3 to 1:10, preferably from 1:0.5 to 1:6, and more preferably 1:1, 1:3, 1:3.75, 1:5 or 1:6.

In one embodiment, in the sustained-release injection preparation of the present invention, the amount of the compound of the formula (I) is 8-22 g/100 ml, the amount of the wetting agent is 0.5-1 g/100 ml, and the amount of the suspending agent is 0.3-5 g/100 ml.

In another embodiment, in the sustained-release injection preparation of the present invention, the amount of the compound of the formula (I) is 8 g/100 ml, 15 g/100 ml, 17 g/100 ml, 20 g/100 ml or 22 g/100 ml, the amount of the wetting agent is 0.5 g/100 ml, 0.6 g/100 ml, 0.8 g/100 ml or 1 g/100 ml, and the amount of the suspending agent is 0.3 g/100 ml, 0.5 g/100 ml or 3 g/100 ml.

In one embodiment, in the sustained-release injection preparation of the present invention, when the wetting agent is Tween 80, the suspending agent is not sodium carboxymethyl cellulose, that is, Tween 80 and sodium carboxymethyl cellulose are not present in the sustained-release injection preparation of the present invention at the same time.

In one embodiment, in the sustained-release injection preparation of the present invention, when the wetting agent is Tween 80 and the suspending agent is sodium carboxymethyl cellulose, the ratio of the wetting agent to the suspending agent is not higher than 1:1.

In one embodiment, in the sustained-release injection preparation of the present invention, when the wetting agent is Tween 80 and the suspending agent is sodium carboxymethyl cellulose, the content of the wetting agent is less than that of the suspending agent.

In one embodiment, in the sustained-release injection preparation of the present invention, when the wetting agent is Tween 80 and the suspending agent is sodium carboxymethyl cellulose, the ratio of wetting agent to suspending agent is 1:1.

In one embodiment, the sustained-release injection preparation of the present invention further comprises a buffer agent.

In another embodiment, in the sustained-release injection preparation of the present invention, the buffer agent is phosphate, preferably disodium hydrogen phosphate, or sodium dihydrogen phosphate.

In one embodiment, in the sustained-release injection preparation of the present invention, R is selected from n-pentyl, n-hexyl, n-heptyl, n-octyl, n-nonyl, n-decyl, n-undecyl, n-tridecyl, n-pentadecyl or N-heptadecyl.

In one embodiment, R is a C₆ alkyl.

In a more preferred embodiment, R is a n-hexyl.

In an embodiment, in the sustained-release injection preparation of the present invention, the compound of formula (I) is selected from the group consisting of:

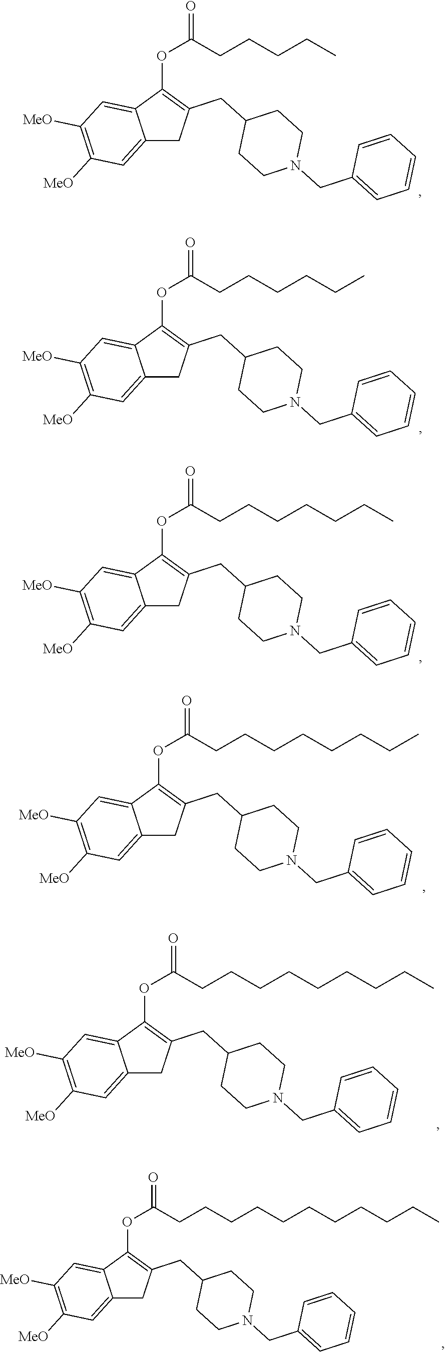 [SMILES: CCCCCC(=O)OC1=C(CC2CCN(Cc3ccccc3)CC2)Cc2cc(OC)c(OC)cc21 ; CCCCCCC(=O)OC1=C(CC2CCN(Cc3ccccc3)CC2)Cc2cc(OC)c(OC)cc21 ; CCCCCCCC(=O)OC1=C(CC2CCN(Cc3ccccc3)CC2)Cc2cc(OC)c(OC)cc21 ; CCCCCCCCC(=O)OC1=C(CC2CCN(Cc3ccccc3)CC2)Cc2cc(OC)c(OC)cc21 ; CCCCCCCCCC(=O)OC1=C(CC2CCN(Cc3ccccc3)CC2)Cc2cc(OC)c(OC)cc21 ; CCCCCCCCCCCC(=O)OC1=C(CC2CCN(Cc3ccccc3)CC2)Cc2cc(OC)c(OC)cc21]

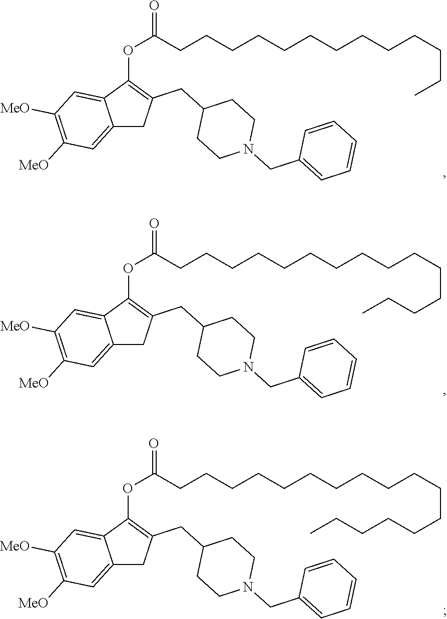 [SMILES: CCCCCCCCCCCCCC(=O)OC1=C(CC2CCN(Cc3ccccc3)CC2)Cc2cc(OC)c(OC)cc21 ; CCCCCCCCCCCCCCCC(=O)OC1=C(CC2CCN(Cc3ccccc3)CC2)Cc2cc(OC)c(OC)cc21 ; CCCCCCCCCCCCCCCCCC(=O)OC1=C(CC2CCN(Cc3ccccc3)CC2)Cc2cc(OC)c(OC)cc21]

and preferably is

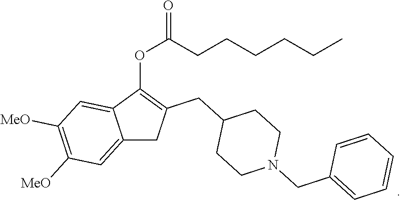 [SMILES: CCCCCCC(=O)OC1=C(CC2CCN(Cc3ccccc3)CC2)Cc2cc(OC)c(OC)cc21]

In another aspect, a sustained-release injection preparation with extended shelf-life is provided in the present invention, wherein the injection preparation comprises a compound of formula (I), a wetting agent and a suspending agent,

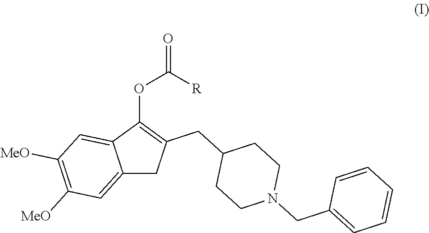

wherein:

R is a C₅₋₁₇ alkyl;

In an embodiment, the suspending agent in the sustained-release injection preparation with extended shelf-life of the present invention is selected from natural polymer suspending agents, synthetic or semi-synthetic polymer suspending agents; preferably one or more selected from the group consisting of gum, celluloses, carbopol, povidone, dextran and polyethylene glycol; more preferably one or more selected from the group consisting of sodium carboxymethyl cellulose (CMC-Na), methyl cellulose, hydroxypropyl cellulose, gum arabic, xanthan gum, povidone and polyethylene glycol; and most preferably, one or more selected from the group consisting of polyethylene glycol 200, polyethylene glycol 400, polyethylene glycol 600, polyethylene glycol 800, polyethylene glycol 1000, polyethylene glycol 1500, polyethylene glycol 4000, polyethylene glycol 6000 and polyethylene glycol 8000.

In one embodiment, the wetting agent in the sustained-release injection preparation with extended shelf-life of the present invention is one or more selected from the group consisting of polysorbates, polyoxyethylene castor oils, poloxamers, tylosamers, povidone, lecithin, polyoxyethylene hydrogenated castor oil, bile salt and polyoxyethylene polyoxypropylene ether block copolymer; and preferably one or more selected from the group consisting of polysorbate 20, polysorbate 40 and polysorbate 80.

In one embodiment, the mixture ratio (weight) of the wetting agent to the suspending agent in the sustained-release injection preparation with extended shelf-life of the present invention is 1:0.3 to 1:10, preferably from 1:0.5 to 1:6, and more preferably 1:1, 1:3, 1:3.75, 1:5 or 1:6.

In one embodiment, in the sustained-release injection preparation with extended shelf-life of the present invention, the content of the compound of the formula (I) is 8-22 g/100 ml, the content of the wetting agent is 0.5-1 g/100 ml, and the content of the suspending agent is 0.3-5 g/100 ml.

In another embodiment, in the sustained-release injection preparation with extended shelf-life of the present invention, the content of the compound of the formula (I) is 8 g/100 ml, 15 g/100 ml, 17 g/100 ml, 20 g/100 ml or 22 g/100 ml, the content of the wetting agent is 0.5 g/100 ml, 0.6 g/100 ml, 0.8 g/100 ml or 1 g/100 ml and the content of the suspending agent is 0.3 g/100 ml, 0.5 g/100 ml or 3 g/100 ml.

In one embodiment, in the sustained-release injection preparation with extended shelf-life of the present invention, when the wetting agent is Tween 80, the suspending agent is not sodium carboxymethyl cellulose, that is, Tween 80 and sodium carboxymethyl cellulose are not present in the sustained-release injection preparation of the present invention at the same time.

In one embodiment, in the sustained-release injection preparation with extended shelf-life of the present invention, when the wetting agent is Tween 80 and the suspending agent is sodium carboxymethyl cellulose, the ratio of the wetting agent to the suspending agent is not higher than 1:1.

In one embodiment, in the sustained-release injection preparation with extended shelf-life of the present invention, the content of the wetting agent is less than that of the suspending agent.

In one embodiment, in the sustained-release injection preparation with extended shelf-life of the present invention, when the wetting agent is Tween 80 and the suspending agent is sodium carboxymethyl cellulose, the ratio of wetting agent to suspending agent is 1:1.

In one embodiment, the sustained-release injection preparation of the present invention further comprises a buffer agent.

In another embodiment, in the sustained-release injection preparation of the present invention, the buffer agent is phosphate, preferably disodium hydrogen phosphate, or sodium dihydrogen phosphate.

In one embodiment, in the sustained-release injection preparation of the present invention, R is selected from n-pentyl, n-hexyl, n-heptyl, n-octyl, n-nonyl, n-decyl, n-undecyl, n-tridecyl, n-pentadecyl or N-heptadecyl.

In one embodiment, in the sustained-release injection preparation of the present invention, the compound of formula (I) is selected from the group consisting of:

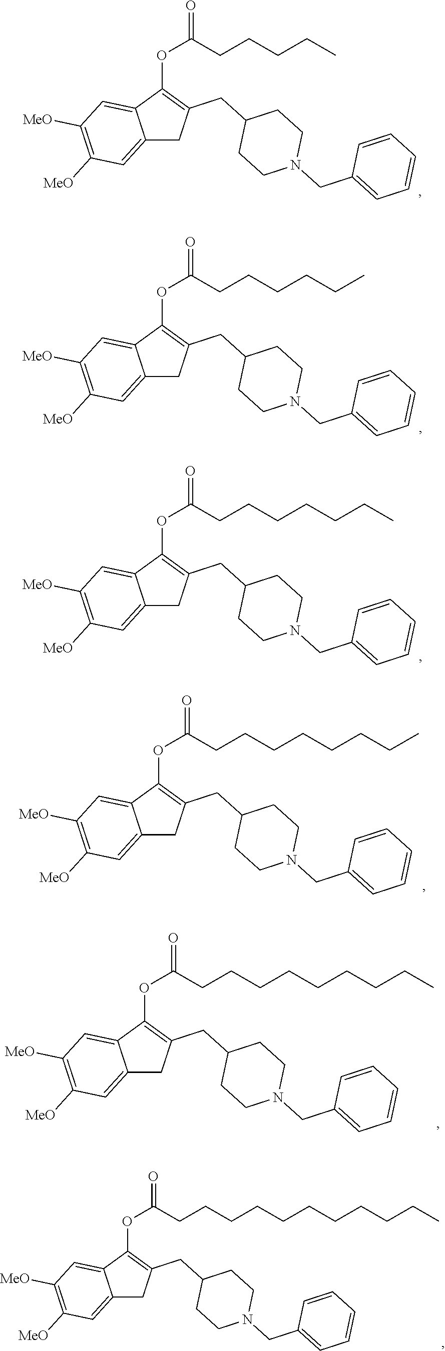 [SMILES: CCCCCC(=O)OC1=C(CC2CCN(Cc3ccccc3)CC2)Cc2cc(OC)c(OC)cc21 ; CCCCCCC(=O)OC1=C(CC2CCN(Cc3ccccc3)CC2)Cc2cc(OC)c(OC)cc21 ; CCCCCCCC(=O)OC1=C(CC2CCN(Cc3ccccc3)CC2)Cc2cc(OC)c(OC)cc21 ; CCCCCCCCC(=O)OC1=C(CC2CCN(Cc3ccccc3)CC2)Cc2cc(OC)c(OC)cc21 ; CCCCCCCCCC(=O)OC1=C(CC2CCN(Cc3ccccc3)CC2)Cc2cc(OC)c(OC)cc21 ; CCCCCCCCCCCC(=O)OC1=C(CC2CCN(Cc3ccccc3)CC2)Cc2cc(OC)c(OC)cc21]

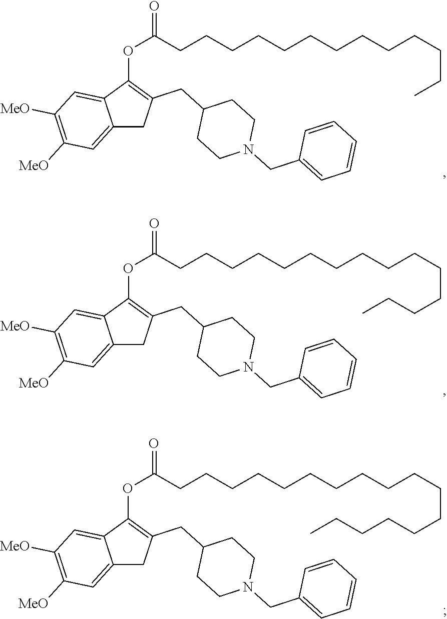 [SMILES: CCCCCCCCCCCCCC(=O)OC1=C(CC2CCN(Cc3ccccc3)CC2)Cc2cc(OC)c(OC)cc21 ; CCCCCCCCCCCCCCCC(=O)OC1=C(CC2CCN(Cc3ccccc3)CC2)Cc2cc(OC)c(OC)cc21 ; CCCCCCCCCCCCCCCCCC(=O)OC1=C(CC2CCN(Cc3ccccc3)CC2)Cc2cc(OC)c(OC)cc21]

and preferably, is

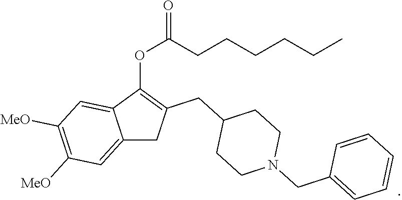 [SMILES: CCCCCCC(=O)OC1=C(CC2CCN(Cc3ccccc3)CC2)Cc2cc(OC)c(OC)cc21]

The sodium dihydrogen phosphate in the present invention includes anhydrous sodium dihydrogen phosphate, sodium dihydrogen phosphate monohydrate, and sodium dihydrogen phosphate dihydrate, and all forms can be used in the preparation of the present invention.

The compound of formula (I) of the present invention is prepared by the following method

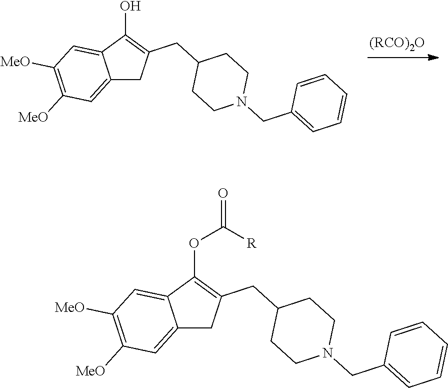

R is a C₅₋₁₇ alkyl.

The Donepezil used for preparing the compound of formula (I) can be commercially purchased or prepared according to known methods.

The present invention also provides a method for treating diseases caused by abnormal acetylcholinesterase activities using the sustained-release injection preparation containing donepezil derivatives of the present invention, including a step of administering a therapeutic amount of the aforementioned sustained-release injection preparation to a patient in need thereof. The preparation can be intramuscularly or subcutaneously administered. The disease is Alzheimer's disease.

The terms used in the present invention will have following meanings, unless otherwise specified.

The term indicating the number of carbon atoms in a group, for example, C₁₋₁₀, means that the group can contain 1 carbon atom, 2 carbon atoms, 3 carbon atoms, etc., up to 10 carbon atoms; and the term “or” can be used interchangeably with the terms “and/or”, unless the context clearly dictates otherwise.

“Alkyl” represents a saturated aliphatic hydrocarbon group with the number of carbon atoms, including straight-chain and branched-chain hydrocarbon groups, including but not limited to methyl, ethyl, propyl, isopropyl, n-butyl, isobutyl, tert-butyl Base, n-pentyl, n-hexyl, etc.

The suspending agent refers to an additive which can increase the viscosity of a dispersion medium to reduce the sedimentation speed of particles or increase the hydrophilicity of the particles, including natural polymer suspending agents, synthetic or semi-synthetic polymer suspending agents, including but not limited to one or more selected from the group consisting of gum, cellulose, carbopol, povidone, dextran and polyethylene glycol. It may be one or more selected from the group consisting of sodium carboxymethyl cellulose, methyl cellulose, hydroxypropyl cellulose, gum arabic, xanthan gum, povidone and polyethylene glycol.

The wetting agent refers to an additive which can increase the wetting of hydrophobic drug particles by water, including one or more selected from the group consisting of polysorbates, polyoxyethylene castor oil, poloxamers, tylosamer, povidone, lecithin, polyoxyethylene ethylene hydrogenated castor oil, bile salt and polyoxyethylene polyoxypropylene ether block copolymer, including but not limited to one or more selected from the group consisiting of polysorbate 20, polysorbate 40 and polysorbate 80. In a preferred embodiment, the wetting agent used in the present invention is polysorbate 80.

The term “therapeutic amount” as used in the present invention refers to an amount leading to an improvement in any parameter or clinical symptom. The actual dosage may vary from patient to patient, and does not necessarily refer to the total amount which eliminates all symptoms of the disease.

“Extended shelf life” as described in the present invention refers to a period during which the aggregation and sedimentation of particles won't occur after the sustained-release injection suspension of donepezil derivatives is stored at room temperature (for example, 25° C.) or a higher temperature (for example, 40° C. or higher). The extended shelf life is at least 1 month, preferably at least 2-4 months, more preferably at least 5-6 months, and more preferably at least 6 months.

### DESCRIPTION OF DRAWINGS

FIG. 1 shows the concentration-time curve of donepezil in the plasma of rats after a suspension of donepezil heptanoate is administered via intramuscular (i.m.) and subcutaneous (s.c.) injection;

FIG. 2 shows the concentration-time curve of donepezil in the plasma of rats after a suspension and oil solution of donepezil heptanoate is administered via intramuscular (i.m.) injection;

FIG. 3 shows the concentration-time curve of donepezil and donepezil caprylate in the plasma of beagle dogs after a suspension of donepezil caprylate is administered via intramuscular injection (i.m.) injection;

FIG. 4 shows the concentration-time curve of donepezil and donepezil heptanoate in the plasma of beagle dogs after a suspension of donepezil heptanoate is administered via intramuscular injection (i.m.) injection.

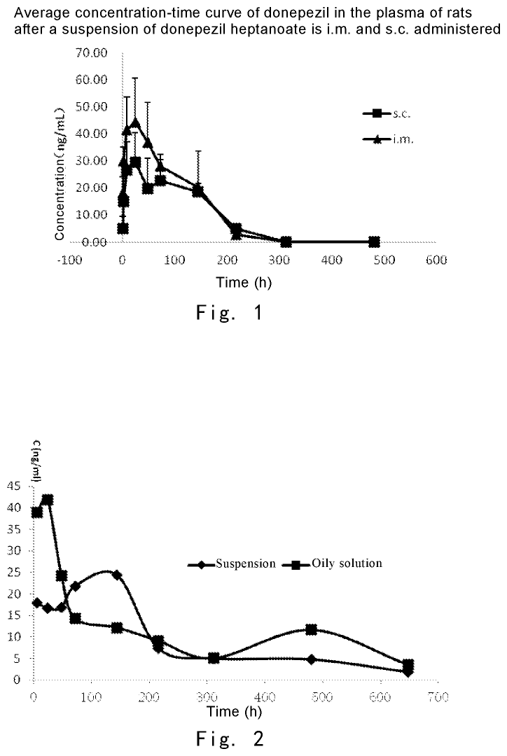

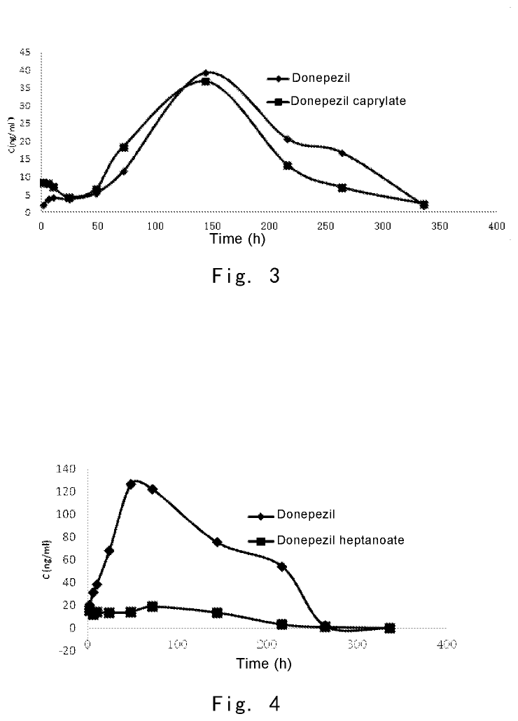

### MODES FOR CARRYING OUT THE INVENTION

After extensive and in-depth research, the inventor unexpectedly discovered that the sustained-release injection in the form of a suspension made of donepezil derivatives, especially specific donepezil derivatives, can possess an excellent stability, re-dispersibility and pharmacokinetic properties, thereby completing the present invention.

“Donepezil derivative” used in the present invention is a compound having the structure of formula (I),

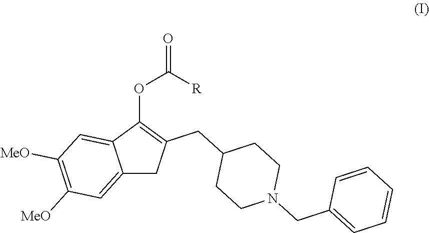

wherein R is a C₅₋₁₇ alkyl.

In a specific embodiment, the “donepezil derivative” of the present invention is donepezil heptanoate; that is, R is n-hexyl.

A skilled person know that it is a commonly used modification method to form an ester for designing a prodrug, however, the major problem for the ester prodrug is that it is difficult to predict pharmacokinetic distribution properties of an ester prodrug. Moreover, the biotransformation of an alkyl ester in human blood is relatively slow and incomplete, so that the bioavailability of an ester prodrug is lower than expected. Therefore, which ester prodrug can exhibit excellent pharmacokinetic properties cannot be reasonably predicted. Furthermore, a skilled person can prepare various forms of sustained-release injections from compounds with pharmacological activities, however, which form of sustained-release injection can exhibit excellent pharmacokinetic properties cannot be reasonably predicted.

The invention discloses a sustained-release injection preparation in the form of a suspension and a preparation method thereof. A skilled person can appropriately improve process parameters based on the contents herein. In particular, it should be noted that all similar substituents and modifications are obvious to those skilled in the art, all of which shall be deemed as being included in the present invention. The method of the present invention and applications thereof have been described through the preferred embodiments. It is obvious that a skilled person can appropriately modify or change and combine the method described herein and applications thereof without departing from the content, spirit and scope of the present invention to achieve and apply the technology of the present invention.

Following preparation examples are provided for explaining the present invention in detail. However, the scope of the present invention is not limited to these examples.

### Example 1. Preparation of the Compound of Formula (I)

Preparation of Compound 1, Donepezil Heptanoate

Donepezil (40.0 g, 105.4 mmol) was added into a 1000 ml three-neck round bottom flask (equipped with argon protection, thermometer, mechanical stirring, constant pressure dropping funnel). The flask was purged with nitrogen, and 300 ml of anhydrous tetrahydrofuran was added and stirred to dissolve the Donepezil. Then the reaction system was cooled to −60˜−78° C. Lithium bis(trimethylsilyl)amide (100 ml, 1.0 mol/L, 100 mmol) was quickly added into the constant pressure dropping funnel through a double-ended needle in one time, and stirred at −60˜−78° C. for 15˜30 mins. The system was naturally warmed up to 0˜10° C. Afterwards, the temperature of the system was lowered to −60˜−78° C., and then heptanoic anhydride (38.82 g, 242.4 mmol) was dissolved in 100 ml of anhydrous tetrahydrofuran and quickly added to the constant pressure dropping funnel in one time, stirred at −60˜−78° C. for 30 mins, and then naturally warmed to room temperature (20-30° C.). TLC detection showed that the raw material was completely consumed. The reaction system was placed in an ice-water bath, and 250 ml of saturated ammonium chloride solution was added dropwise. Upon addition, the liquid was separated, and the obtained organic phase was washed with 250 ml of 20% sodium chloride solution and 250 ml of saturated sodium chloride solution, and separated. The organic phase was dried over anhydrous sodium sulfate, and the solvent was removed under reduced pressure to obtain 29.3 g of an oily material (yield: 93%). The oily material was heated and dissolved in 225 ml of n-heptane and filtered to remove insolubles. And then 22.5 ml of ethanol was added and cooled. Crystals precipitated at −5˜−10° C. for 1 h. The crystals were was obtained by filteration, washed with 50 ml of cold n-heptane/ethanol (10:1), suction-dried, and dried under vacuum to obtain 42.30 g of off-white solids (yield: 81.7%).

HPLC(aera): 98.2%.

Mass spectrometry (m/z): [M+H]⁺=492.4.

¹H-NMR (CDCl₃) δ: 7.32-7.31 (4H, d), 7.26-7.27 (1H, m), 6.98 (1H, s), 6.59 (1H, s), 3.89 (3H, s), 3.90 (3H, s), 3.50 (2H, s), 3.26 (2H, s), 2.87-2.89 (2H, d), 2.60-2.64 (2H, t), 2.27-2.29 (2H, d), 1.90-1.93 (2H, t), 1.80-1.83 (2H, t), 1.65-1.68 (2H, t), 1.20-1.60 (9H, m), 0.90-0.96 (3H, t).

### Example 2. Preparation of Suspension

Formulation

| | | Wetting | Suspending | Dibasic Sodium | | Citric |
|---|---|---|---|---|---|---|
| | Compound 1 | agent | agent | Phosphate | Mannitol | acid |
| Formulation 1 | 8.0% | 0.5% Tween80 | 0.5% | 200 mM | / | / |
| | | | CMC-Na | pH 6.75 | | |
| Formulation 2 | 8.0% | 0.5% Tween80 | 3.0% | 200 mM | / | / |
| | | | PEG4000 | pH 6.75 | | |
| Formulation 3 | 8.0% | 1.0% Tween80 | 3.0% | 200 mM | / | / |
| | | | PEG4000 | pH 6.75 | | |
| Formulation 4 | 22% | 0.8% Tween80 | 3.0% | 50 mM | 4.0% | / |
| | | | PEG4000 | pH 7.0 | | |
| Formulation 5 | 20% | 1.0% Tween80 | 0.5% | 50 mM | 4.0% | / |
| | | | CMC-Na | pH 5.5 | | |
| Formulation 6 | 20% | 0.5% Tween20 | 3.0% | 50 mM | 4.0% | / |
| | | | PEG4000 | pH 7.0 | | |
| Formulation 7 | 20% | 0.6% Tween80 | 3.0% | 50 mM | 4.0% | / |
| | | | PEG4000 | pH 7.0 | | |
| Formulation 8 | 20% | 0.5% Tween20 | 0.5% | 50 mM | 4.0% | / |
| | | | CMC-Na | pH 5.5 | | |
| Formulation 9 | 20% | 1.0% Tween80 | 3.0% | 50 mM | 4.0% | / |
| | | | PEG4000 | pH 7.0 | | |
| Formulation 10 | 20% | 0.8% Tween80 | 3.0% | 50 mM | 4.0% | 0.5% |
| | | | PEG4000 | pH 7.0 | | |
| Formulation 11 | 20% | 1.0% Tween80 | 0.3% | 50 mM | 4.0% | / |
| | | | CMC-Na | pH 5.5 | | |
| Formulation 12 | 20% | 0.8% Tween80 | 3.0% | 50 mM | 4.0% | 0.5% |
| | | | PEG4000 | pH 7.0 | | |
| Formulation 13 | 17% | 0.8% Tween80 | 3.0% | 50 mM | / | 0.5% |
| | | | PEG4000 | pH 7.0 | | |
| Formulation 14 | 15% | 1.0% Tween80 | 3.0% | 50 mM | / | 0.5% |
| | | | PEG4000 | pH 7.0 | | |
| Formulation 15 | 15% | 0.8% Tween80 | 3.0% | 50 mM | / | 0.5% |
| | | | PEG4000 | pH 7.0 | | |

X % means that every 100 ml of suspension contains X g. For example, 0.5% Tween 80 means that 0.5 g of Tween 80 is contained in 100 ml of suspension.

Preparation Method

1) Excipients, such as the wetting agent, suspending agent, buffer (dibasic sodium phosphate) were dissolved in an appropriate amount of injectable water, the pH was adjusted with dilute phosphoric acid or sodium hydroxide, if necessary, and the solution was filtered and sterilized;

2) Compound 1 was added into the solution obtained in step 1), and homogeneously dispersed;

3) the material obtained in step 2 was processed to an appropriate particle size (ball mill or homogenization), and water for injection was added to adjust to the target concentration.

### Example 3. Stability and Redispersibility of the Particle Size of Compound 1 in Suspension

The size of particles in a suspension is not only related to the quality and stability of the suspension, but also affects the efficacy and bioavailability of the suspension. Therefore, the determination of the particle size and distribution thereof in a suspension is an important index for evaluating the quality of the suspension.

For a good suspension, if it is shaken after storage, the precipitates should be quickly re-dispersed, so as to ensure the uniformity when administered.

Malvern 3000 laser particle size analyzer was used to measure the particle size of the sample, expressed in volume particle size. D10 refers to a particle size when the cumulative particle size distribution percentage of a sample reaches 10%, and the unit of particle size is micron. The same applies to D50 and D90. The sample was shaken for 30 seconds before measurement so as to make the particles in the suspension evenly dispersed. A sample was takedn and added into the sample cell (dispersion medium was purified water), and the data (D10/D50/D90) was read after the obscuration was stable. If the sample cannot be shaken to completely dispersed, it is indicated by “aggregation”.

At 25° C., the particle size of Compound 1 in suspension was measured at Day 0 and Day 30.

| | 25° C., Day 0 | 25° C., Day 30 |
|---|---|---|
| Formulation 1 | 0.596/1.46/3.18 | NA |
| Formulation 2 | 0.596/1.53/3.66 | 0.574/1.51/3.52 |
| Formulation 3 | 0.577/1.46/3.27 | 0.569/1.48/3.36 |
| Formulation 4 | 0.70/2.24/6.42 | 0.753/2.26/6.47 |
| Formulation 5 | 0.70/2.18/6.07 | 0.769/2.24/6.07 |
| Formulation 6 | 0.67/2.01/5.53 | 0.708/2.09/5.87 |
| Formulation 7 | 0.68/2.24/6.34 | 0.705/2.20/6.38 |
| Formulation 8 | 0.67/1.94/5.14 | 0.730/2.15/5.78 |
| Formulation 13 | 0.907/3.22/9.06 | 0.777/2.48/6.88 |

NA means “not detected”

At 40° C., the particle size of Compound 1 in suspension was measured at Day 0, Day 10 and Day 30.

| | 40° C., Day 0 | 40° C., Day 10 | 40° C., Day 30 |
|---|---|---|---|
| Formulation 1 | 0.596/1.46/3.18 | 0.606/1.51/4 | 0.628/1.71/53.2 |
| Formulation 2 | 0.596/1.53/3.66 | 0.598/1.53/3.82 | 0.609/1.64/5.92 |
| Formulation 3 | 0.577/1.46/3.27 | 0.612/1.5/3.26 | 0.633/1.62/4.01 |
| Formulation 4 | 0.70/2.24/6.42 | 0.786/2.38/6.93 | 0.818/2.44/7.17 |
| Formulation 5 | 0.70/2.18/6.07 | 0.78/2.27/6.5 | Aggregation |
| Formulation 6 | 0.67/2.01/5.53 | 0.744/2.2/6.46 | 0.744/2.20/6.46 |
| Formulation 7 | 0.68/2.24/6.34 | 0.753/2.22/6.53 | 0.771/2.42/8.86 |
| Formulation 8 | 0.67/1.94/5.14 | 0.768/2.2/6.01 | 0.811/2.61/8.68 |
| Formulation 13 | 0.907/3.22/9.06 | 0.818/2.66/7.36 | 0.790/2.53/6.94 |

At 60° C., the particle size of Compound 1 in suspension was measured at Day 0, Day 5, Day 10 and Day 30.

| | 60° C., Day 0 | 60° C., Day 10 | 60° C., Day 30 |
|---|---|---|---|
| Formulation 1 | 0.596/1.46/3.18 | 0.620/1.53/3.87 | 0.703/2.43/166 |
| Formulation 2 | 0.596/1.53/3.66 | 0.6/1.5/3.43 | 0.629/1.79/8.97 |
| Formulation 3 | 0.577/1.46/3.27 | 0.698/1.72/3.68 | 0.732/2/4.58 |
| Formulation 4 | 0.70/2.24/6.42 | 0.912/2.82/8.35 | 1.05/3.95/11 |
| Formulation 5 | 0.70/2.18/6.07 | 0.912/2.94/9.49 | Aggregation |
| Formulation 6 | 0.67/2.01/5.53 | 0.956/3.15/7.97 | 1.19/4.78/15.0 |
| Formulation 7 | 0.68/2.24/6.34 | 0.859/2.69/7.91 | 1.05/4.41/19.1 |
| Formulation 8 | 0.67/1.94/5.14 | 0.888/2.68/7.12 | 1.08/3.75/15.2 |
| Formulation 13 | 0.907/3.22/9.06 | 0.890/3.29/9.52 | 1.39/6.90/17.3 |

Re-dispersibility: it can be seen from the test results that the suspension containing Tween 80 and CMC-Na is easy to agglomerate to form large particles, and it is not easy to be dispersed after being shaken.

### Example 4. Pharmacokinetic Study

Experiment 1

6 male SD rats (weight of 220-230 g) were randomly divided into 2 groups (3 rats in each group). One group was intramuscularly injected with 30 mg/kg of donepezil heptanoate suspension (Formulation 14). Another group was given an equal dosage of donepezil heptanoate suspension (Formulation 14) by subcutaneous injection. Approximately 0.5 h, 2 h, 8 h, 24 h (d2), 48 h (d3), 72 h (d4), 144 h (d7), 216 h (d10), 312 h (d14) and 480 h (d21) after administration, approximately 0.3 mL of each blood sample was collected into a 1.5 mL centrifuge tube containing sodium fluoride and potassium oxalate in advance for anticoagulation, and temporarily stored on ice until centrifugation. The whole blood was centrifuged to collect plasma, which was transferred to a 96-well plate, and stored at −20° C. for detection by LC-MS/MS. The drug concentration at each sampling time was determined by measuring the standard curve of reference donepezil in plasma.

According to the statistical moment theory, relevant pharmacokinetic parameters corresponding to compound concentrations in SD rats at each time point after i.m. and s.c. administration of donepezil heptanoate were calculated respectively, using WinNonlin 6.3 software. See Table-1 for details.

TABLE 1
Pharmacokinetic parameters of donepezil in plasma of rats after i.m.
and s.c. injection of 30 mg/kg donepezil heptanoate suspension

| | i.m. | s.c. |
|---|---|---|
| T_{1/2}(h) | 52.9 | 53.6 |
| Tₘₐₓ(h) | 11.3 | 16.7 |
| Cₘₐₓ(ng/mL) | 46.9 | 32.6 |
| AUCₗₐₛₜ(h*ng/mL) | 5270 | 4146 |
| AUC_{INF}_{_{—}}_{obs}(h*ng/mL) | 5467 | 4437 |
| MRTₗₐₛₜ(h) | 74.8 | 92 |

It can be seen from the test results that, after the injection of the present invention was administered at the same dosage, the AUC of intramuscular (i.m.) administration is higher than the AUC of subcutaneous (s.c.) administration, which is about 1.3 times that of subcutaneous administration, that is, intramuscular administration exhibits better absorption capacity than subcutaneous administration.

Experiment 2

6 male SD rats (weight of 220-230 g) were randomly divided into 2 groups (3 rats in each group). One group was intramuscularly injected with 90 mg/kg of donepezil heptanoate suspension, and another group was intramuscularly injected with an equal dosage of donepezil heptanoate oil solution. Approximately 6 h, 24 h (d2), 48 h (d3), 72 h (d4), 144 h (d7), 216 h (d10) and 312 h (d14) after administration, approximately 0.3 mL of each blood sample was collected into a 1.5 mL centrifuge tube containing sodium fluoride and potassium oxalate in advance for anticoagulation, and temporarily stored on ice until centrifugation. The whole blood was centrifuged to collect plasma, which was transferred to a 96-well plate, and stored at −20° C. for detection by LC-MS/MS. The drug concentration at each sampling time was determined by measuring the standard curve of reference donepezil in plasma. See Table 2 and Figure-2 for specific data.

TABLE 2
Concentration of donepezil in plasma of rats at
each sampling time after i.m. administration of
donepezil heptanoate suspension and oil solution

| Time Hour | Suspension (ng/mL) | Oil solution (ng/mL) |
|---|---|---|
| 6 h | 17.96 | 39 |
| 24 h (d 2) | 16.66 | 41.86 |
| 48 h (d 3) | 16.79 | 24.24 |
| 72 h (d 4) | 21.82 | 14.25 |
| 144 h (d 7) | 24.35 | 12.16 |
| 216 h (d 10) | 7.32 | 9.06 |
| 312 h (d 14) | 4.98 | 5.04 |

Formulation of suspension: 0.5% CMC-Na, 5% Mannitol, 0.5% Tween 80, 3% PEG4000; formulation of Oil solution: Benzyl benzoate/Castor oil = 36/21 (w/w).

It can be seen from the test results that, compared with the oil solution, there is no sudden release phenomenon for the suspension of the present invention after administration, and the blood concentration curve is relatively stable.

Experiment 3

6 male non-naïve beagle dogs weighing 8.6-10.6 kg used in this example were purchased from Beijing Marshall Biotechnology Co., Ltd. All of the animals were over 1 year old, and there was an interval of at least two weeks from the last test.

There were 3 animals in each group. The animals were intramuscularly injected with donepezil caprylate suspension (18.4 mg/kg, calculated in donepezil, prepared according to donepezil heptanoate in Example 1) and donepezil heptanoate suspension (23.8 mg/kg calculated in donepezil). Approximately 2 h, 6 h, 10 h, 24 h (d2), 48 h (d3), 72 h (d4), 144 h (d7), 216 h (d10), 264 h (d12) and 336 h (d15) after administration, approximately 0.3 mL of each blood sample was collected into a 1.5 mL centrifuge tube containing sodium fluoride and potassium oxalate in advance for anticoagulation, and temporarily stored on ice until centrifugation. The whole blood was centrifuged to collect plasma, which was transferred to a 96-well plate, and stored at −20° C. for detection by LC-MS/MS. The drug concentration at each sampling time was determined by measuring the standard curve of reference donepezil in plasma. See Table-3, Figure-3 and Figure-4 for specific data.

TABLE 3
Concentrations of donepezil and prodrug in plasma of rats
at each sampling time after i.m. administration of donepezil
heptanoate suspension and donepezil caprylate suspension

| | Donepezil heptanoate | | Donepezil caprylate | |
|---|---|---|---|---|
| | suspension (ng/mL) | | suspension (ng/mL) | |
| | | Donepezil | | Donepezil |
| Time Hour | Donepezil | heptanoate | Donepezil | caprylate |
| 2 h | 20.38 | 15.07 | 1.15 | 4.29 |
| 6 h | 31.36 | 11.78 | 2.37 | 4.00 |
| 10 h | 38.46 | 14.04 | 2.98 | 3.28 |
| 24 h(d 2) | 68.04 | 13.67 | 3.07 | 2.86 |
| 48 h(d 3) | 126.32 | 14.31 | 4.80 | 3.95 |
| 72 h(d 4) | 121.89 | 19.04 | 8.99 | 10.53 |
| 144 h(d 7) | 75.35 | 13.8 | 60.08 | 45.47 |
| 216 h(d 10) | 54.26 | 3.29 | 19.80 | 11.65 |
| 264 h (d 12) | 2.14 | 0.99 | 15.16 | 5.43 |
| 336 h (d 15) | N/A | N/A | 2.05 | 2.35 |

Formulation of Donepezil caprylate suspension: 1.5% Tween 80, 4% PEG4000, 4% Mannitol;
Formulation of Donepezil heptanoate suspension: 0.5% Tween 80, 0.5% CMC-Na, 5% Mannitol (90 mg/ml).

According to the test results, it can be seen that after administration, donepezil heptanoate can be rapidly metabolized into donepezil, therefore, the risk of side effects caused by the prodrug is extremely low.

The above are only the preferred embodiments of the present invention. It should be noted that a skilled person can make improvements and modifications without departing from the principle of the present invention, and these improvements and modifications shall be regarded as falling within the protection scope of the present invention.

1. A sustained-release injection preparation in the form of a suspension, wherein the injection preparation comprises a compound of formula (I), a wetting agent and a suspending agent, 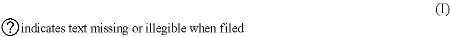
wherein:
R is a C₅₋₁₇ alkyl.

2. The sustained-release injection preparation of claim 1, wherein the wetting agent is one or more selected from the group consisting of polysorbates, polyoxyethylene castor oils, poloxamers, tylosamers, povidone, lecithin, polyoxyethylene hydrogenated castor oil, bile salt and polyoxyethylene polyoxypropylene ether block copolymer; and preferably one or more selected from the group consisting of polysorbate 20, polysorbate 40 and polysorbate 80; and more preferably, polysorbate 80.

3. The sustained-release injection preparation of claim 1, wherein the suspending agent is selected from natural polymer suspending agents, synthetic or semi-synthetic polymer suspending agents; preferably one or more selected from the group consisting of gums, celluloses, carbopol, povidone, dextran and polyethylene glycol; more preferably one or more selected from the group consisting of sodium carboxymethyl cellulose, methyl cellulose, hydroxypropyl cellulose, gum arabic, xanthan gum, povidone and polyethylene glycol; and most preferably, one or more selected from the group consisting of polyethylene glycol 200, polyethylene glycol 400, polyethylene glycol 600, polyethylene glycol 800, polyethylene glycol 1000, polyethylene glycol 1500, polyethylene glycol 4000, polyethylene glycol 6000 and polyethylene glycol 8000.

4. The sustained-release injection preparation of claim 1, wherein the mixture ratio (weight) of the wetting agent to the suspending agent is 1:0.3 to 1:10, preferably from 1:0.5 to 1:6, and more preferably 1:1, 1:3, 1:3.75, 1:5 or 1:6.

5. The sustained-release injection preparation of claim 1, wherein the amount of the compound of the formula (I) is 8-22 g/100 ml, the amount of the wetting agent is 0.5-1 g/100 ml, and the amount of the suspending agent is 0.3-5 g/100 ml.

6. The sustained-release injection preparation of claim 5, wherein the amount of the compound of the formula (I) is 8 g/100 ml, 15 g/100 ml, 17 g/100 ml, 20 g/100 ml or 22 g/100 ml, the amount of the wetting agent is 0.5 g/100 ml, 0.6 g/100 ml, 0.8 g/100 ml or 1 g/100 ml, and the amount of the suspending agent is 0.3 g/100 ml, 0.5 g/100 ml or 3 g/100 ml.

7. The sustained-release injection preparation of claim 1, wherein the sustained-release injection preparation of the present invention further comprises a buffer agent.

8. The sustained-release injection preparation of claim 7, wherein the buffer agent is phosphate, preferably disodium hydrogen phosphate, or sodium dihydrogen phosphate.

9. The sustained-release injection preparation of claim 1, wherein R is selected from n-pentyl, n-hexyl, n-heptyl, n-octyl, n-nonyl, n-decyl, n-undecyl, n-tridecyl, n-pentadecyl or N-heptadecyl.

10. The sustained-release injection preparation of claim 1, wherein R is a C₆ alkyl.

11. The sustained-release injection preparation of claim 10, wherein R is a n-hexyl.

12. The sustained-release injection preparation of claim 1, wherein the compound of formula (I) is selected from the group consisting of:

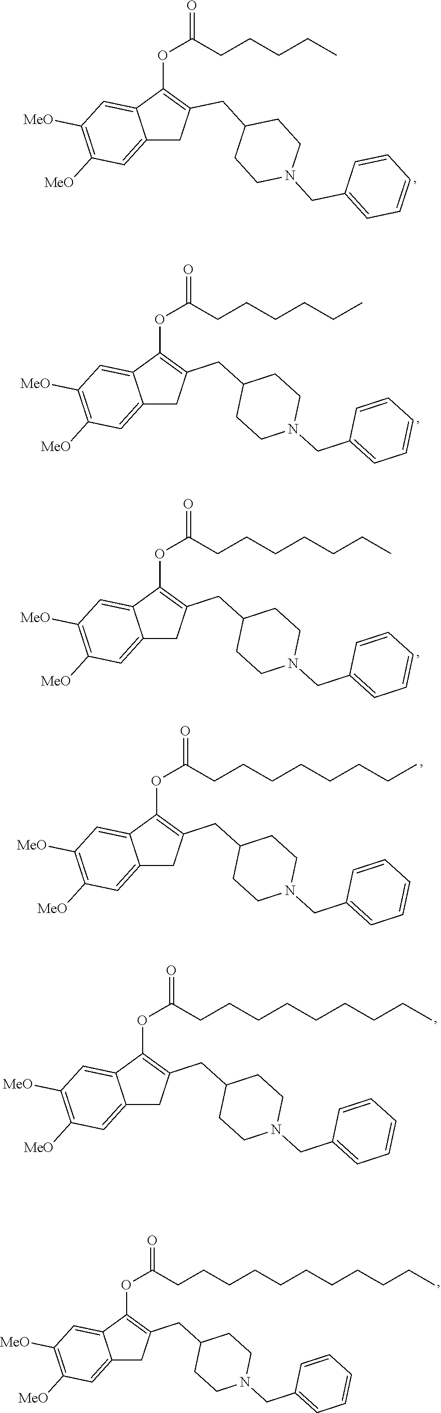 [SMILES: CCCCCC(=O)OC1=C(CC2CCN(Cc3ccccc3)CC2)Cc2cc(OC)c(OC)cc21 ; CCCCCCC(=O)OC1=C(CC2CCN(Cc3ccccc3)CC2)Cc2cc(OC)c(OC)cc21 ; CCCCCCCC(=O)OC1=C(CC2CCN(Cc3ccccc3)CC2)Cc2cc(OC)c(OC)cc21 ; CCCCCCCCC(=O)OC1=C(CC2CCN(Cc3ccccc3)CC2)Cc2cc(OC)c(OC)cc21 ; CCCCCCCCCC(=O)OC1=C(CC2CCN(Cc3ccccc3)CC2)Cc2cc(OC)c(OC)cc21 ; CCCCCCCCCCCC(=O)OC1=C(CC2CCN(Cc3ccccc3)CC2)Cc2cc(OC)c(OC)cc21]

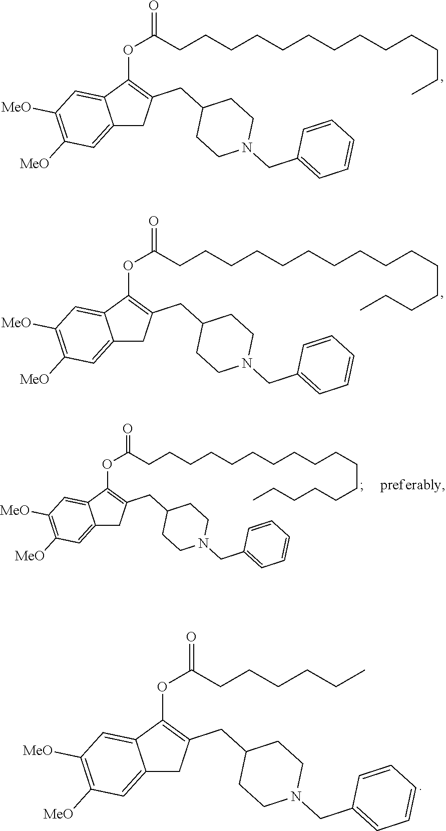 [SMILES: CCCCCCCCCCCCCC(=O)OC1=C(CC2CCN(Cc3ccccc3)CC2)Cc2cc(OC)c(OC)cc21 ; CCCCCCCCCCCCCCCC(=O)OC1=C(CC2CCN(Cc3ccccc3)CC2)Cc2cc(OC)c(OC)cc21 ; CCCCCCCCCCCCCCCCCC(=O)OC1=C(CC2CCN(Cc3ccccc3)CC2)Cc2cc(OC)c(OC)cc21 ; CCCCCCC(=O)OC1=C(CC2CCN(Cc3ccccc3)CC2)Cc2cc(OC)c(OC)cc21]

13. The sustained-release injection preparation of claim 1, wherein the period during which the aggregation and sedimentation of particles won't occur after the sustained-release injection suspension of donepezil derivatives is stored at 25° C. or 40° C. is at least 1 month; preferably at least 2-4 months; more preferably at least 5-6 months; and more preferably at least 6 months.

14. Use of the sustained-release injection preparation of claim 1 in the preparation of a medicament for treating diseases caused by abnormal acetylcholinesterase activity.

15. The use of claim 14, wherein the diseases is Alzheimer's disease.

16. The use of claim 14, wherein the medicament is intramuscularly or subcutaneously administered; and preferably, intramuscularly administered.

17. The sustained-release injection preparation of claim 1, wherein the sustained-release injection preparation is intramuscularly or subcutaneously administered; and preferably, intramuscularly administered.
